(19) United States
(12) Patent Application Publication
(10) Pub. No.: US 2008/0146493 A1
(43) Pub. Date: Jun. 19, 2008

(54) Use of Haptoglobin and Hemoglobin to Modulate Na Ion Transport in a Vertebrate

(76) Inventors: Hugh O'Brodovich, Oakville (CA); Michael Harris, Lausanne (CH); Umadevi Sajjan, Ann Arbor, MI (US); Bijan Rafii, Richmond Hill (CA); Don Mahuran, Toronto (CA); Gail Otulakowski, Thornhill (CA); Alex Hillar, Mississauga (CA)

Correspondence Address: MYERS BIGEL SIBLEY & SAJOVEC, PO BOX 37428, RALEIGH, NC, 27627, US

(21) Appl. No.: 11/632,975

(22) PCT Filed: Jul. 19, 2005
(86) PCT No.: PCT/CA2005/001136

§ 371 Date: Aug. 30, 2007

Related U.S. Application Data
(60) Provisional application No. 60/588,755, filed on Jul. 19, 2004.

(51) Int. Cl. A61K 38/42; A61K 38/16; C07K 14/805; C07K 14/435

(52) U.S. Cl. 514/6; 514/12; 530/385; 530/380; 435/4

(57) ABSTRACT

The invention provides methods and compositions for increasing Na ion transport and/or epithelial fluid transport in a vertebrate by administering an effective amount of haptoglobin or haptoglobin and hemoglobin. The invention also provides a new therapeutic approach for treating disorders associated with insufficient epithelial Na ion and/or fluid transport, the use of an effective amount of haptoglobin, or haptoglobin and hemoglobin in the preparation of medicament for increasing epithelial Na ion transport in a vertebrate as well as a method of screening a candidate compound for its ability to modulate haptoglobin or haptoglobin and hemoglobin stimulation of epithelial Na ion transport.

### FIELD OF THE INVENTION

The invention relates to methods for increasing Na ion and fluid transport and more specifically to use of haptoglobin or haptoglobin and hemoglobin to increase such transport.

### BACKGROUND OF THE INVENTION

Lung edema is caused by high transvascular pressure gradients as seen in congestive heart failure (CHF), increased permeability to solutes as occurs in the adult respiratory distress syndrome (ARDS), or neonatal respiratory distress syndrome (n-RDS) or a combination of the two. It has been assumed that edema fluid (EF) was removed from the airspaces via hydrostatic and osmotic pressure gradients. In both high-pressure and high-permeability pulmonary edema, however, there is a substantial amount of protein within the alveolar fluid that opposes osmotic reabsorption. Indeed, passive forces cannot explain the rate of alveolar fluid clearance (AFC) from the intact lung (Matthay et al., 1982). AFC arises from the distal lung epithelial (DLE) amiloride-sensitive and amiloride-insensitive active Na⁺ transport, with Cl⁻ and water following (Matthay et al., 2002; Matalon et al., 1999; O'Brodovich et al., 2004). Epithelial Na ion transport is rate limited by the number of active Na ion permeant ion channels in the epithelial apical membrane and is often associated with a concomitant increase in the Na⁺/K⁺ ATPase activity (Rafii et al., 2002).

Epithelial Na ion transport represents an important homeostatic process in many tissues and aberrations in this transport play a role in a number of diseases. Pulmonary edema occurs in CHF, as well as in ARDS, and improved survival and clinical parameters correlate with intact active fluid absorption from the airspace (Verghese et al., (1999); Ware et al., (2001)). At birth, the lungs' airspaces are cleared of fetal lung fluid by Na ion transport and if this transport is inadequate, respiratory distress or even death can occur (Hummler et al., (1996); O'Brodovich et al., (2004)).

Prior to the 1970's, the occurrence of significant respiratory distress in a newborn infant carried a grave prognosis with an approximate 75% mortality rate during the first days of life. This rapidly changed as understanding of newborn lung physiology improved. It was discovered that there was a relative deficiency of airspace surface active material in the condition, then termed Idiopathic Respiratory Distress Syndrome, and assisted ventilation became possible. Together these advances soon led physicians to recognize that there were actually two common forms of non-infective respiratory distress in the newborn human infant. One became known as hyaline membrane disease (HMD) or neonatal respiratory distress syndrome (nRDS). The other became known as transient tachypnea of the newborn (TTN). Further work determined that both nRDS and TTN are characterized by increased lung water content, although only the former has inadequate amounts of surfactant, and that inadequate active epithelial Na⁺ transport plays an important pathophysiologic role in the initiation of both of these disorders (O'Brodovich et al., 2004). Lung epithelial Na ion and fluid transport occurs by both amiloride-sensitive and amiloride-insensitive pathways. Optimal activity of the amiloride blockable epithelial Na ion channel (EnaC) and other Na ion permeant ion channels is also critical to the normal function of the lungs' airways and also to the function of other organs, including the kidney and gastrointestinal tract.

Other diseases, such as cystic fibrosis, are characterised by abnormally elevated airway epithelial Na ion transport (Knowles et al., (1981)), with resultant dehydration of the airway surface, and defective mucociliary clearance. The important role of both amiloride sensitive and amiloride insensitive Na ion transport is illustrated by the fact that CF has an increase in amiloride insensitive Na and fluid transport and that CF respiratory tract epithelia, in contrast to normal human respiratory epithelia, still exhibit significant fluid absorption after they are treated with amiloride; indeed, amiloride-treated CF epithelia absorb fluid at or close to the same rate as untreated normal human respiratory epithelia (Jiang et al., (1993)).

Excessive renal Na ion reabsorption also occurs in the kidney in systemic hypertension, and gain of function mutations in ENaC cause Liddle's disease (Shimkets et al., (1994)). In contrast, loss of function ENaC gene defects cause the severe salt wasting nephropathy, pseudohypoaldosteronism (Chang et al., (1996)). Ulcerative colitis, a chronic inflammatory disease of the intestinal tract, has also been shown to be associated with reduced amiloride-sensitive Na ion transport (Amasheh et al., 2004).

Intact AFC correlates with survival and important clinical parameters, such as the length of assisted ventilation and oxygen requirements, regardless of whether the patient suffers from acute CHF- or ARDS-induced pulmonary edema (Matthay et al., 1990; Verghese et al., 1999; Ware et al., 2001).). Notably, only 75% of patients with CHF-induced pulmonary edema have demonstrable AFC and only ˜40% of patients are able to achieve maximal AFC rates (Verghese et al., supra). The situation is even graver for patients with acute lung injury and ARDS; less than 15% of these patients can achieve maximal AFC rates and AFC is impaired more frequently in men and when there is associated sepsis (Ware et al., supra).

Although animal studies have shown that exogenous catecholamines can augment AFC when left atrial pressures are elevated (Campbell et al, 1999), patient studies have not shown a correlation between the levels of circulating catecholamines and AFC (Verghese and Ware, supra). Other agents, such as growth factors, cytokines, dopamine and steroids, increase AFC in the normal lung (Matthay et al., 2002) but their effect on AFC in CHF or ARDS or n-RDS patients is unknown. To date, there are no therapeutic agents available that have been proven to increase AFC in patients with pulmonary edema.

The ability to effectively modulate Na ion transport in vivo has been very limited to date. There remains a need for methods and compositions which can modulate Na ion transport.

### SUMMARY OF THE INVENTION

Haptoglobin, or haptoglobin and hemoglobin, have been shown to stimulate epithelial Na ion and fluid transport. This provides a new approach to treating disorders associated with insufficient epithelial Na ion and/or fluid transport.

The involvement of haptoglobin or haptoglobin and hemoglobin in epithelial Na ion transport further provides a new therapeutic approach to conditions such as hypertension where Na ion transport is excessive by blocking the effect of haptoglobin or haptoglobin and hemoglobin on such transport.

In accordance with one embodiment of the present invention, there is provided a method of increasing epithelial Na ion transport and/or epithelial fluid transport in a vertebrate by administering to the vertebrate an effective amount of haptoglobin or an effective amount of haptoglobin and hemoglobin.

In accordance with another embodiment of the present invention, there is provided a pharmaceutical composition comprising haptoglobin or haptoglobin and hemoglobin.

In accordance with a further embodiment of the present invention, there is provided use of an effective amount of haptoglobin or an effective amount of haptoglobin and hemoglobin in the preparation of a medicament for increasing epithelial Na ion transport in a vertebrate.

In accordance with a further embodiment of the present invention, there is provided a method for screening a candidate compound for its ability to modulate haptoglobin or haptoglobin and hemoglobin stimulation of epithelial Na ion transport comprising:

providing an in vitro cell system which carries out Na ion transport;

contacting the cell system with haptoglobin or haptoglobin and hemoglobin in the presence or absence of the candidate compound; and

determining Na ion transport in the cell system;

wherein a different level of Na ion transport in the presence of the compound compared with the level in the absence of the compound indicates that the compound modulates the haptoglobin or haptoglobin and hemoglobin stimulation epithelial of Na ion transport.

### SUMMARY OF THE DRAWINGS

Certain embodiments of the invention are described, reference being made to the accompanying drawings, wherein:

FIG. 1 shows AFC (fluid cleared %. h⁻¹) in rats 24 hours after lung instillation of Ringer's lactate (RL, n=16, P<0.01), bovine serum albumin in Ringer's lactate (BSA, N=5, P<0.05) or pulmonary edema fluid (EF, n=15).

FIG. 2A shows ATII cell apical membrane density, as number of channels per patch (number) of highly Na⁺ selective cation channels (Na⁺ Channel, *P<0.01, n=14-20) and non-selective cation channels (NSC) after treatment with EF (solid bars) or control medium (open bars);

FIG. 2B shows open probability (Po) of highly Na⁺ selective channels (Na Channel) and non-selective channels (NSC) in the ATII cells of FIG. 2A after treatment with EF (solid bars) or control medium (open bars);

FIG. 3 shows % control amiloride-insensitive short circuit current (Isc) (open bars) and amiloride sensitive Isc (hatched bars) in rat-DLE cells (r-DLE) and human airway epithelial cells (HBE) treated with rat pulmonary EF (r-EF, n=20), rat globulin (r-Gl, n=15) or human globulin (h-Gl, n=8);

FIG. 4A shows the effect on amiloride-insensitive short circuit current (AIC) in rat-DLE cells of various fractions obtained by DEAE ion exchange chromatography of rat globulin (open bars). Effect of unpurified globulin is shown as solid bar.

FIG. 4B shows the specific activity of pooled HPLC fractions of rat globulin on AIC in rat-DLE cells;

FIG. 5A shows % decrease from control in AIC of rat-DLE cells by rat globulins treated with varying amounts of anti-Hp antibody (HpAb);

FIG. 5B shows AIC in rat-DLE cells treated with rat Hp protein immunopurified from globulins (IP-Hp) or with a control preparation from globulins using non-specific rat IgG (n=6,*P<0.01);

FIG. 5C shows AIC in human bronchial cells treated with various concentrations of commercially available human Hp;

FIG. 6A shows the effect on AIC in rat-DLE cells of rat hemoglobin (Hb), rat haptoglobin (Hp), rat Hp/Hb complex (Cx) and medium alone (M);

FIG. 6B shows the effect of ion transport inhibitors on Na ion transport (short circuit current Isc) stimulated by various concentrations of Hp/Hb complex (Cx).

### DETAILED DESCRIPTION OF THE INVENTION

The present invention provides a method of increasing epithelial Na ion transport in a vertebrate by administering to the vertebrate an effective amount of haptoglobin or of haptoglobin and hemoglobin. As epithelial fluid transport is driven by active Na ion transport, the method of the invention provides a method of increasing epithelial fluid transport, as well as Na ion transport.

In particular, haptoglobin or haptoglobin and hemoglobin have been shown to increase Na ion and fluid transport. In lung epithelial cells, this increased Na ion transport has been shown to be due to an increased number of active Na⁺ permeant ion channels in the apical membranes with a concomitant increase in the Na⁺/K⁺ ATPase activity.

There is thus provided a method for treating, in a vertebrate, a disorder associated with reduced Na ion and fluid transport or associated with fluid accumulation in excess of Na ion and fluid transport capacity.

There is also provided a composition comprising haptoglobin or haptoglobin and hemoglobin for increasing epithelial Na ion and fluid transport in a vertebrate.

The subunit composition of epithelial Na ion transport channels may vary from tissue to tissue or there may be different Na ion channels from tissue to tissue, resulting in differing sensitivity to amiloride inhibition. Regardless of whether the Na ion transport channel which is defective in any particular disease is amiloride-sensitive or not, an increase in Na ion and fluid transport in any channel in the relevant organ will be beneficial by removing unwanted fluid.

Epithelial Na ion and fluid transport is important, for example, in the lungs, the kidneys and the gastro-intestinal tract. It is known that humans who have different haptoglobin polymorphisms (phenotypes) have different sensitivities to dietary sodium, different predispositions to systemic hypertension, different responses to antihypertensive medications and different prevalences of coronary artery disease (Langlois et al., 1996).

In one embodiment, the invention provides a method of increasing lung epithelial Na ion transport in a vertebrate by administering to the vertebrate an effective amount of haptoglobin or haptoglobin and hemoglobin. The treated vertebrate may be a bird or an animal, for example a mammal, including a human.

This provides a method for treating a lung disorder associated with reduced Na ion transport or excessive fluid accumulation. Such disorders include congestive heart failure-induced pulmonary edema, adult respiratory distress syndrome or combinations thereof, neonatal respiratory distress syndrome (hyaline membrane disease) and transient tachypnea of the newborn and similar disorders.

In further embodiments, the invention provides a method for treating ulcerative colitis and kidney diseases associated with reduced Na ion and fluid transport such as pseudohypoaldosteronism (Chang et al., (1996) or other renal tubular disorders.

An initial observation that mammalian edema fluid was able to produce an increase in Na ion transport in mammalian lung-derived cells led to the recognition that one component of the edema fluid, a hemoglobin/haptoglobin complex, causes increased AFC in vivo in the rat lung and increased Na ion transport in human and rat lung epithelial cells. Haptoglobin and hemoglobin together, and haptoglobin alone, have been shown to produce increased epithelial Na ion transport.

Haptoglobin, an α₂ acid glycoprotein, is an acute phase reactant protein whose main function appears to be to be to scavenge hemoglobin, which, in its free state, is toxic to endothelium and epithelium. Haptoglobin has a high binding affinity for hemoglobin, kd=1×10⁻¹⁵ mol/L.

In humans, haptoglobin consists of combinations of α¹, α² and β chains. There are three phenotypes, Hp (1-1) which has the structure (α¹β)₂, Hp (2-1) which has the structure (α¹β)₂ (α²α)ₙ following a linear polymeric series where n is 1 or greater, and Hp (2-2) which has the structure (α²β)ₙ, also a polymeric series where n is 3 or greater (Kurosky et al., 1980; Dobrszycka, 1997).

When haptoglobin and hemoglobin interact, it appears that an Hp/Hb complex is formed by the binding of at least one, and up to two, hemoglobin α/β dimers with one molecule of haptoglobin.

In the methods and compositions of the invention, one may use haptoglobin or haptoglobin and hemoglobin. The haptoglobin and hemoglobin may be used in any proportions, the haptoglobin being complexed partially or fully with hemoglobin.

Haptoglobin and hemoglobin for use in the methods and compositions of the invention can be prepared using commercially available hemoglobin and haptoglobin.

For use in human therapy, human hemoglobin and haptoglobin are used. Purified human hemoglobin is commercially available, for example, from Sigma Chemicals.

Commercially available hemoglobin may be subject to oxidation during purification, leading to the formation of meth-hemoglobin. It should be reduced before use. The hemoglobin is therefore treated, for example, in solution, with sodium dithionite while nitrogen gas is bubbled through the solution, to reduce the hemoglobin to the ferrous state, followed by bubbling CO gas through the solution for a short period of time to convert the hemoglobin to its carbonmonoxyferrous liganded form, which is suitable for stable storage or for combining with haptoglobin to form Hb/Hp complex.

Human haptoglobin, prepared from pooled plasma, is also commercially available, either as a mixture containing all three haptoglobin phenotypes or as a preparation of a single Hp phenotype. Purified human haptoglobin preparations are available, for example, from Sigma Chemicals and Life Diagnostics. The most typical trace impurity in haptoglobin preparations is hemoglobin, which does not present a problem when the haptoglobin is used to prepare an Hb/Hp complex. Any of these forms of haptoglobin may be used.

Those of skill in the pharmaceutical art will be aware of final purification procedures and methods of formulation applicable to human proteins such as an Hp or an Hb/Hp complex to be used for therapy.

Alternatively, haptoglobin may be synthesized by recombinant techniques. Hp cDNA sequences, for example human cDNA (gi51093872) are obtainable through the databanks such as GenBank. Thus clones may be prepared by RT-PCR from total RNA preparations and propagated in common bacterial or other vectors. Recombinant Hp may then be expressed by transfection of an appropriate vector containing the Hp cDNA sequence into a cell line capable of carrying out N-linked glycosylation of the Hp polypeptide (e.g. insect Sf9 cells, mammalian cell lines; for example, see Heinderyckx et al., 1989). To aid in purification of recombinant Hp, the cDNA sequence may be preceded or immediately followed in the same reading frame to incorporate a polyhistidine tag or other sequence for the same purpose. Following determination of optimized expression conditions for the Hp protein in the chosen cell line, Hp is then purified and isolated using standard techniques which can include, but are not limited to, affinity chromatography on an immobilized nickel binding resin (e.g. BD Talon resin), ion exchange chromatography, gel filtration chromatography, or other chromatography or bulk techniques.

An Hp/Hb complex is formed by combining the above-described hemoglobin and haptoglobin, for example in a one to one molar ratio.

Hp or an Hb/Hp complex can be screened for activity to stimulate Na ion transport using an in vitro rat distal lung epithelial cell system as described in the Examples herein, using rat edema fluid as positive control and medium as negative control, and studying the cells in Using chambers as described herein.

Hp or an Hb/Hp complex may be quantitated either by an ELISA assay (Life Diagnostics Inc.) or via a method exploiting the peroxidase activity of Hb when fully complexed to Hp (Phase™ haptoglobin spectrophoto-metric assay, Tri-Delta Diagnostics Inc., U.S. Pat. No. 6,451,550). It is within the skill of the medical professional involved to determine an appropriate dosage for therapy.

The type of formulation and method of administration required is dependent on the disorder to be treated. For example, for treatment of a lung disease, as described above, haptoglobin or haptoglobin and hemoglobin may be administered by instillation in aqueous solution, optionally including a pharmaceutically acceptable surfactant, or by administration of the solution as an inhaled aerosol, by a device such as a spritzer, nebuliser or metered dose inhaler.

For other diseases, such as kidney or gastrointestinal tract diseases, the complex may be administered by intravenous, oral or rectal administration, as appropriate.

It is known that humans with Hp 1-1 and 2-1 phenotypes are susceptible to salt-sensitive hypertension (Kojima et al., 1994; Weinberger et al., 1987) and the Hp 2-2 phenotype is associated with refractory hypertension, which cannot be controlled by standard drug therapy (Delanghe et al., 1995).

Hp has been identified as a highly homologous member of the serine protease family, although it lacks a functional proteolytic “catalytic triad” (Kurosky, 1980). Serine proteases have been identified as in vivo regulators of Na ion transport in various tissues, including the lung and the kidney (Vallet et al., 1997; Vuagniaux et al., 2000; Donaldson et al., 2002).

It is postulated that Hp or Hp and Hb can modulate epithelial Na ion transport in the kidney and that Hp and/or Hp and Hb are involved in salt-sensitive hypertension. This provides a new therapeutic approach for treatment of hypertension, by administering a compound which modulates the effect of Hp or Hp and Hb on kidney Na ion transport.

The invention further provides a screening method for identifying such compounds, by examining the effect of candidate compounds on Hp or Hp and Hb stimulation of Na ion transport in kidney epithelial cells. For example, kidney cell lines such as A6 and Ml could be employed and the assay carried out essentially as in the examples described herein, using Ussing Chambers.

A suitable cell or tissue preparation which carries out. Na ion transport is treated with Hp or Hp and Hb in the presence or absence of a candidate compound, and compounds are thereby identified which can modulate the effect of Hp or Hp and Hb on Na ion transport. Compounds which reduce the effect of Hp or Hp and Hb on Na ion transport are potential therapeutic compounds for the treatment of hypertension.

### EXAMPLES

The examples are described for the purposes of illustration and are not intended to limit the scope of the invention.

Methods of molecular biology, protein and peptide biochemistry and immunology referred to but not explicitly described in this disclosure and examples are reported in the scientific literature and are well known to those skilled in the art.

### Materials and Methods

Reagents Reagents were obtained from Sigma Chemicals, Oakville, ON (amiloride, Protein G-agarose, human haptoglobin, globulins, albumin and hemoglobin), Life Diagnostics, Inc., West Chester, Pa. (rat haptoglobin); Immunology Consultants Lab. Inc., Newberg, Oreg. (affinity purified rabbit anti-rat haptoglobin IgG), Accurate Chemical and Scientific Corp., (Westbury, N.Y. (anti-human haptoglobin IgG), Biogenesis Ltd., Kingston, N.H. (antibody to rat hemoglobin), Bio-Rad Laboratories Ltd., Mississauga, ON (macroprepDEAE support, 4-15% gradient gels, alkaline phosphatase anti-rabbit IgG); and Gibco, Burlington, ON; (culture media, FBS).
Animal protocols. The protocols for all animal experiments were reviewed and approved by the Hospital for Sick Children Animal Care committee.
Preparation of edema fluid (EF) and plasma from rats. EF and Plasma preparation from rats were done according to protocols published previously (Rafii et al., (2002)). Briefly, four hundred gram male Sprague Dawley rats were anesthetized using ketamine (80 mg/kg) and xylazine (80 mg/kg). A tracheostomy was performed and assisted ventilation (F₁O₂=1.0, tidal volume of 5 ml, 35/min) was commenced. Next, after a thoracotomy, the aorta was clamped for 30 sec after infusion of 15 ml/kg Ringers solution through the penile vein and EF was collected through an endotracheal catheter. The EF was centrifuged at 10,000 g for 30 min at 4° C. Plasma was obtained by centrifuging heparinized blood at 1,000 g for 15 minutes at 4° C. Plasma and the EF supernatant were stored in aliquots at −85° C. EF was diluted 1:1 with DMEM for Ussing chamber experiments
Cell culture methods. All cells were maintained at 37° C. with 5% CO₂ balanced humidified air.
Bioelectric Measurements in cell monolayers. Monolayers of r-DLE and HBE were studied in Ussing chambers (Rafii et al., 2002) at 37° C. under short circuit conditions while bathed in Hank's balanced salt solution (Invitrogen, Burlington, ON), with transepithelial potential difference (PD) determined intermittently using voltage/current clamps (VCC600, Physiologic Instruments, San Francisco, Calif.). Apical amiloride (10⁻⁴M) yielded amiloride-sensitive and -insensitive short circuit current (I_{sc}) Primary monolayer cultures of lung epithelia were exposed to EF or protein preparations for 18-24 h prior to being studied in Ussing chambers.

ATII were studied using cell-attached patch-clamp configurations at room temperature (RT) (Chen et al., 2002). Cell-attached bath and pipette solutions contained (in mM): NaCl(140), MgCl(1), CaCl₂(1), KCl(5), HEPES(10), pH=7.4 with 2N—NaOH. Current amplitude histograms were made from stable continuously-recorded data, and the open and closed current levels determined from least-square-fitted gaussian distributions. The number of channels (N) times single channel open probability (Pₒ) indicated patch channel activity. The total number of a patch's functional channels (N) was estimated by observing the number of peaks in current-amplitude histograms constructed, and when possible from long-event records to provide 95% confidence of the correct N (Marunaka et al., 0.1991). The effect of EF was determined after 18 h exposure to these agents on the basolateral (HBE, ATII) or apical/basolateral sides (r-DLE). This approach has been previously used to determine the number of active Na⁺ permeant ion channels in the DLE (Chen et al., 2002; Marunaka et al., 1991; Compeau et al., 1994).

Globulin Fractionation Rat globulins in 20 mM Tris HCl(pH=8.0) were fractionated on macroprep DEAE support using stepwise increases in NaCl concentrations. Biologically active fractions were further separated by preparative reverse-phase HPLC employing μBondapak-C18 column (125 Å poresize, 10 μm particle size, 7.8 mm×300 mm; (Waters Ltd. Mississagua, ON) with a linear gradient of 30-80% acetonitrile/water/0.1% trifluoroacetic acid. Fractions from multiple runs were pooled, concentrated and dialyzed (DMEM) for functional assays. Unfractionated globulin was used at 3.5 mg/ml in DMEM.
In-gel tryptic digestion and mass spectrometry. Biologically active fractions from the HPLC purification step underwent SDS-PAGE, protein bands were visualized (Coomassie blue R-250), cut out and trypsin digested (She et al., 2002). Tryptic peptides underwent peptide fingerprinting and MS/MS sequencing (Applied Biosystems/MDS Sciex API QSTAR XL MALDI QTOF). Peptide fingerprinting of the in-gel digests was analyzed by database searching with ProFound (129.85.19.192/profound_bin/WebProFound.exe) and the peptide sequence obtained by MS/MS spectra was interpreted manually and searched for protein homologies (www.ncbi.nim.nih.gov; 80/BLAST).
Depletion of Hp from globulins. Rat globulins (3 ml of 10 mg/ml) were incubated with 25-600 μg of either antihuman-Hp IgG or non-specific IgG overnight at 4° C. After 1 h incubation at room temperature with Protein G-agarose, the supernatant fraction was dialyzed with DMEM and diluted 1:1 with culture medium for functional assays.
Hp Immunopurification from globulins. Polyclonal anti-human Hp was crosslinked (according to manufacturer's instructions) to protein G-agarose using Immunopure® Protein G-IgG Plus Orientation kit (Pierce biotechnology, Rockford Ill.). Globulins (100 mg/6 ml PBS) dialyzed against PBS (0.1M phosphate buffer pH=7.2 containing 0.15M NaCl) were incubated with the immobilized antibody at 4° C. overnight, unbound fraction eluted and washed with 2×PBS. Bound material was eluted with 0.1M glycine-HCl (pH=2.8) and immediately neutralized with Tris HCl. Eluted fractions were dialyzed against DMEM for functional assays.
SDS-PAGE and western blot analysis Proteins underwent SDS-PAGE under reduced conditions on gels that were stained using Coommassie blue or were transferred to nitrocellulose or PVDF membrane for western analysis. Blots were blocked with 3% BSA, incubated with affinity purified rabbit anti-rat Hp (Immunology Consultants Lab. Inc., Newberg, Oreg.), anti-rat Hb (Cedarlane, Hornby, ON) or anti-human Hb (Cortex Biochem, San Leandro, Calif.), and unbound antibody was removed by washing (TTBS, 10 mM Tris/0.15M NaCl/0.05% Tween 20). Bound antibody was detected with conjugated alkaline phosphatase anti-rabbit IgG using NBT-BCIP (Roche Diagnostics, Laval, QC).
Statistical analysis To test the difference between group means, Student's t-tests or analysis of variance (including Tukey's test for post hoc analysis) were used. In cases where the parametric assumptions of normality and/or equality of variance between the groups were not met, appropriate non-parametric tests were used instead. Type I error of 0.05 was used as the threshold to recognize statistically significant difference between group means.

### Haptoglobin Quantitation

Total Hp in samples was determined using both a radial immunodiffusion method (Life Diagnostics Inc.) and a colorimetric assay method based on the peroxidase activity of the Hp-Hb complex (Phase™ Assay Kit, Tridelta Diagnostics Inc., Morris Plains, N.J.).

### Hemoglobin Quantitation

The amount of Hb that was present in the commercially available Hp preparations was quantitated using the cyanomethemoglobin spectrophotometric method described by Choudhri et al., 1997, with the exceptions that freshly prepared Drabkin's reagent was used and hemoglobin standard and dilutions thereof were prepared using lyophilized rat hemoglobin (Sigma) dissolved in 50 mM Tris-HCl, pH 8.0.

### Preparation of Rat Haptobglobin-Hemoglobin Complexes In Vitro

Commercial lyophilized rat Hb (Sigma) was suspended in PBS at a concentration of about 4 mg/ml, the remaining insolubles being removed by brief centrifugation (5 min, 4000 g), and the Hb suspension was purged with nitrogen gas. About 10 mg of crystalline sodium dithionite were added directly to the Hb in order to reduce the heme iron and the solution was desalted (Sephadex G25). The Hb solution was briefly (approx. 30-60 s) bubbled with air to ensure conversion to the oxyHb form, subsequently confirmed spectrophotometrically. The oxyHb solution was then assayed for protein concentration via the Bio-Rad protein microassay procedure. Purified rat Hp (Life Diagnostics) was added to the prepared oxyHb solution at a 1:1.1 molar stoichiometry in a screw capped test tube and allowed to incubate overnight at 4° C. with gentle agitation. The Hp-Hb complex solution was then subjected to concentration and buffer replacement (×3) with DMEM medium using an Ultrafree 100 centrifugal concentration filter (Millipore Corp.), according to the manufacturer's instructions.

### Example 1

Alveolar Fluid Clearance (AFC) measurements. AFC measurements were made in anesthetized (80 mg/kg ketamine, 8 mg/kg xylozine) male Sprague-Dawley rats (317±3.6 grams) by instilling 0.5 ml of either Ringers-lactate, EF or bovine serum albumin (BSA, 18 gm/dl) into each lung under fluoroscopic guidance. One day later, rats were re-anesthetized (85 mg/kg pentobarbital sodium) and ventilated (F₁O₂=1; 40-50/min; tidal volume=8 ml/kg) through an endotracheal tube. Blood pressure, heart rate, and blood gases were monitored via a carotid arterial line. After an initial 30 minute period where the animal was allowed to stabilize, 6 ml/kg of an isotonic FITC-dextran (MW>200 kDa, 0.4 mg dextran/ml, 50 mg/ml BSA) Ringers solution (AFC solution) was instilled into one lung. PaCO₂ was monitored throughout the experiment and maintained at 35-40 torr by adjusting the ventilator rate. Osmolality of AFC solution was adjusted to that of plasma using concentrated saline. AFC (% of solution cleared per h) was calculated from dextran concentrations at t=0 and 30 min (Yue et al., 1997) using the following formula:

ALC=(1−Cᵢ/C_{f})×2

where Cᵢ and C_{f} are the initial and final concentrations of dextran respectively.

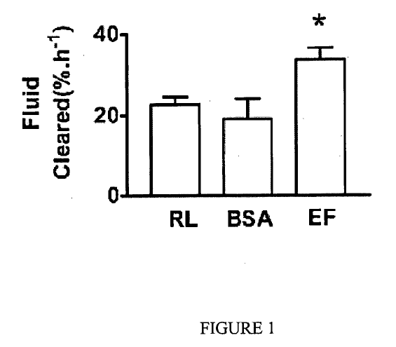

The results are shown in FIG. 1. 24 hours after EF instillation into the lungs of normal rats, the rats had significantly increased AFC. This was not seen in control rats whose lungs were instilled with Ringer-lactate or BSA-Ringers-lactate solutions.

### Example 2

Adult type 11 alveolar (ATII) cells from adult Sprague-Dawley rats were grown at apical air-liquid interface onto permeable culture supports as described previously (Jain et al., 2001). Briefly, the rats were anesthetized with pentobarbital sodium and were heparinized (100 units/kg). ATII cells were digested by tracheal instillation of elastase (0.4 mg/ml). Lung tissue was minced in DNase type IV from bovine pancreas (1 mg/ml; Sigma, St. Louis, Mo.) and filtered sequentially through 100- and 20-μm nylon mesh. The purification was based on differential adherence of cells to dishes coated with rat IgG. Non-adherent ATII cells were collected, centrifuged, and seeded onto Millipore permeable supports and cultured for 24 h submersed in 3 parts Coon's medication of Ham's F-12:7 parts Liebovitz's L-15 with 1 μm dexamethasone. Medium overlying the apical surface was then removed and ATII were cultured with an apical air-liquid interface for an additional 24 hr prior to carrying out patch clamp experiments.

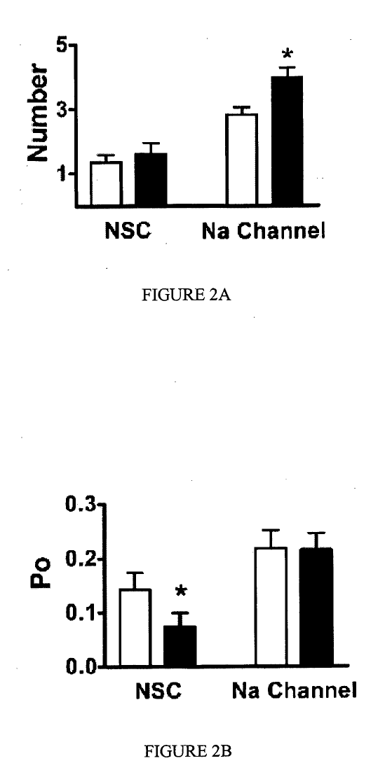

When alveolar type 11 (ATII) cells were exposed to rat edema fluid for 18 h, there was a 1.4±0.15 fold increase (p<0.01, FIG. 2A) in the apical membrane density of the highly selective Na⁺ channels, with no significant change in single channel current or the open probability of these channels (FIG. 2B). At the same time, the open probability of the non-selective channels decreased to 0.52±0.21 (p<0.013, FIG. 2B) of untreated cells, with no significant change in single channel current or the number of non-selective channels.

The major cation channel was highly selective for Na⁺ over K⁺ with a Na⁺:K⁺ permeability ratio (P_{Na}/P_{K})>40 and had a unit conductance of 6 pS with mean open and closed times of 1.23±0.418 s and 3.64±1.23 s respectively. The non-selective channel had a P_{Na}/P_{K} of close to 1, a unit conductance of 21 pS with mean open and closed times of 19±6 ms and 397±172 ms, respectively.

### Example 3

Rat distal lung epithelial (rat-DLE) cells were prepared according to previously published methods (Rafii et al., (2002)). Briefly, lungs from 20d gestation Wistar rat fetuses (Charles River, St. Constant, Quebec; breeding day=day 0, term=22 d) were minced and digested using trypsin. Later this digest was exposed to collagenase and fibroblasts were removed by differential adhesion. DLE were seeded at 1×10⁶ cells/cm² onto 0.4 m pore size Snapwell® cell culture inserts (Corning Costar, Cambridge, Mass.) in DMEM (4.5 g/l glucose with 2 mM L-glutamine and 110 mg/l sodium pyruvate) containing, 10% FBS, 100 U/ml penicillin G sodium, 100 μg/ml streptomycin sulphate and submersion cultured. Twenty four hours later, media was changed to removed non-adherent cells.

Mucociliary-differentiated human tracheobronchial epithelial cell cultures (HBE) were established from cells obtained from normal human trachea or bronchi as described previously (Sajjan et al., 2002) with some modifications. HBE were at first passage seeded (3−5×10⁴ cells/cm²) onto permeable membranes, submerged for 7d in bronchial epithelial cell growth medium (BEGM; Clonetics, San Diego, Calif.) and then switched to air-liquid interface for 14d (1:1 BEGM:DMEM).

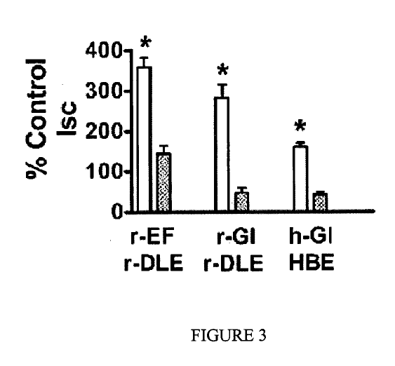

The results are shown in FIG. 3. Confluent monolayers of r-DLE and mucociliary-differentiated HBE showed increased (*P<0.01) AIC (open bars) when incubated overnight with rat pulmonary EF (n=20) or species specific rat (r-Gl n=15) or human (h-Gl, n=8) globulin proteins. No significant change in amiloride-sensitive (cross-hatched bars) Isc occurred. The control group for rat DLE had a transepithelial resistance of 1496±158Ω per cm², baseline Isc of 3.0±0.2 μA/cm² with an AIC of 1.1±0.2 μA/cm². The control group for HBE had a transepithelial resistance of 772±55Ω per cm², base Isc of 30.0±1.9 μA/cm², and AIC of 1.6±0.1 μA/cm².

Both human and rat globulin proteins increased AIC in rat-DLE and HBE cells.

### Example 4

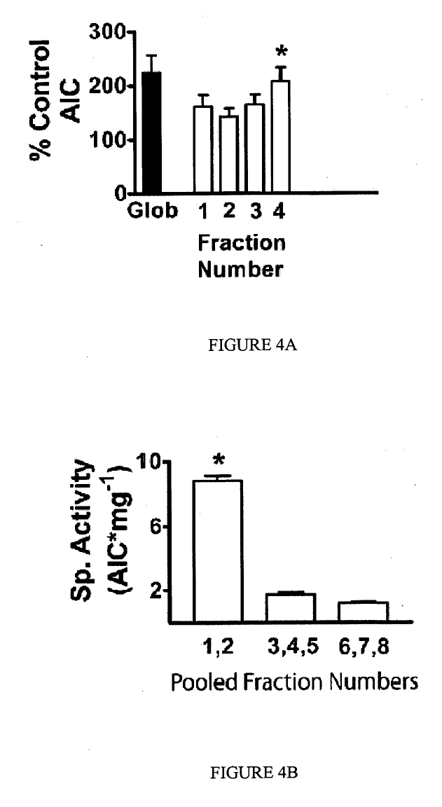

Rat globulin was fractioned by DEAE ion exchange chromatography and fractions eluted by 50 mM, 150 mM and 1.0 M NaCl respectively were tested for their effect on AIC in rat-DLE cells (n=8-11). The results are shown in FIG. 4A. All fractions tended to increase AIC, the 0.1 M NaCl eluate causing a statistically significant AIC increase. This fraction displayed multiple protein bands of molecular weight from <16-200 kDa by SDS-PAGE and was further fractionated by HPLC to yield eight fractions, each containing multiple protein species resolvable by SDS-PAGE. (data not shown). Adjacent fractions were pooled and tested for their effect on the AIC of rat-DLE cells. The results are shown in FIG. 4B. Combined fractions 1 and 2 increased AIC.

Combined fractions 1 and 2 gave 5 bands stainable with Coomassie blue after SDS-PAGE. These bands were further characterized by tryptic digestion and mass spectrometry. Two bands (<16 and ˜45 kDa) contained peptides derived from both haptoglobin (Hp) and hemoglobin (Hb). A 64 kDa band was found to be albumin. Rat Hp contains two subunits, α and β, with predicted molecular weights of 9 and 40 kDa respectively; the β-subunit is glycosylated for an observed MW of ˜45 kDa. Hp is known to bind Hb with very high affinity (kd=1×10⁻¹⁵ mol/L).

### Example 5

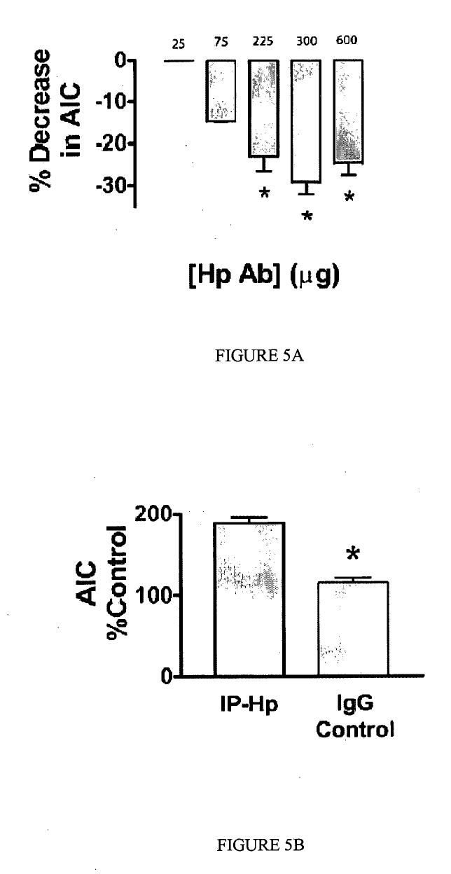

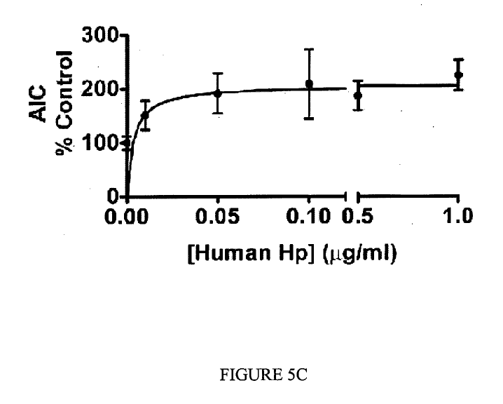

Rat globulins were incubated with varying concentrations of anti-Hp antibody and the antigen-antibody complex was removed by adsorption to protein-G agarose. A dose-dependent abrogation of the globulin's ability to stimulate AIC in rat-DLE cells was observed (FIG. 5A); this was not seen when globulins were incubated with non-specific IgG (data not shown).

Rat globulins were passed through columns containing either anti-Hp IgG or non-specific rabbit IgG coupled to protein G-agarose, unbound proteins were eluted with PBS, and bound proteins were recovered by elution with glycine buffer (pH=2.8). The bound proteins recovered by elution showed a major band of ˜45 kDa, which co-migrated with commercially available rat Hp, and a minor band (˜148 kDa); both reacted with affinity-purified rat anti-Hp antibody indicating that the fraction contained Hp polymers or Hp associated non-covalently with other proteins, such as Hb. The bound fraction eluted from non-specific IgG column showed only two minor bands (˜98 and 148 kDa) and neither reacted with anti-Hp antibody. The Hp immunopurified from rat globulins significantly induced AIC (FIG. 5B) relative to control (bound fraction eluted from the non-specific IgG column). Furthermore, commercially available purified human Hp also stimulated AIC in HBE (FIG. 5C; n=4-9; *P<0.05).

Examination by western blotting showed that EF, immunopurified rat Hp, and commercially available Hp preparations contained Hb, but that rat plasma did not contain Hb (data not shown).

### Example 6

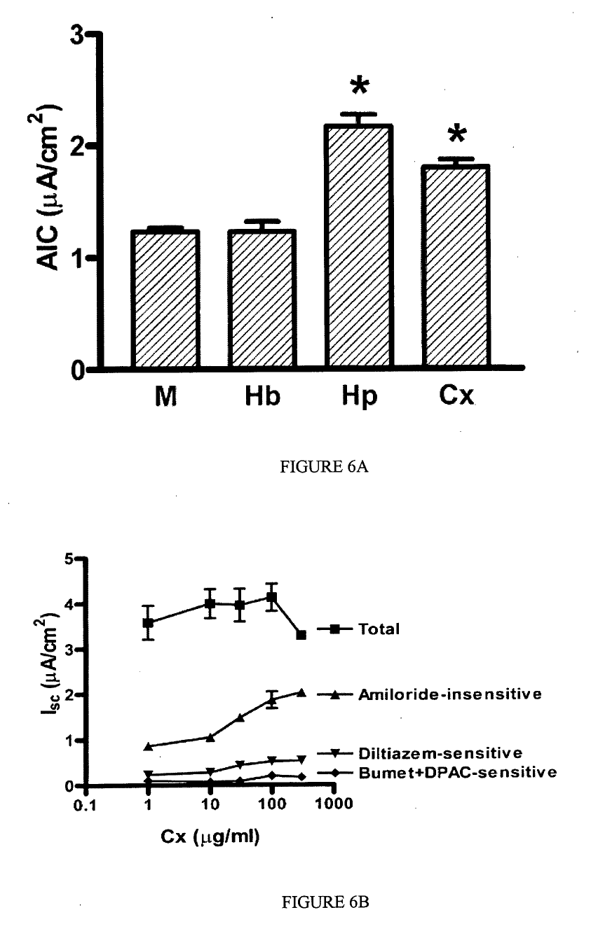

Rat DLE cells were treated with rat hemoglobin, rat haptoglobin or rat Hp/Hb complex and AIC stimulation was determined as described above. The results are shown in FIG. 6A. Both haptoglobin and Hp/Hb complex stimulated Na ion transport.

Rat DLE cells were then treated with various concentrations of commercially available rat Hp and the effect of ion transport inhibitors on the Hp-induced stimulation of Na ion transport was examined. The results are shown in FIG. 6B.

The pattern of inhibition by the ion transport inhibitors used was comparable to that seen on edema fluid induction of AIC.

### REFERENCES

Amasheh et al., 2004, Gastroenterology, v. 126, pp. 1711-1720
Barker, P., Nguyen, M. S., Gatzy, J. T., Grubb, B., Norman, H., Hummler, E., Rossier, B., Boucher, R., and Koller, B. 1998. Role of gammaENaC subunit in lung liquid clearance and electrolyte balance in newborn mice. J. Clin. Invest 102:1634-1640.
Bowler, R. P., Duda, B., Chan E. D, Enghild, J. J., Ware, L. B., Matthay, M., and Duncan, M. W. 2004. Proteomic analysis of pulmonary edema fluid and plasma in patients with acute lung injury. Am. J. Physiol Lung Cell Mol. Physiol 286:L1095-L1104.
Campbell, A. R., Folkesson, H. G., Berthiaume, Y., Gutkowska, J., Suzuki, S., and Matthay, M. A. 1999. Alveolar epithelial fluid clearance persists in the presence of moderate left atrial hypertension in sheep. J. Appl. Physiol. 86:139-151.
Canessa, C. M., Horisberger, J. D., and Rossier, B. C. 1993. Epithelial sodium channel related to proteins involved in neurodegeration. Nature 361:467-470.
Canessa, C. M., Schild, L., Buell, G., Thorens, B., Gautschi, I., Horisberger, J.-D., and Rossier, B. C. 1994. Amiloride-sensitive epithelial Na⁺ channel is made of three homologous subunits. Nature 367:463-467.
Chang, S. S., Grunder, S., Hanukoglu, A., Rosler, A., Mathew, P. M., Hanukoglu, I., Schild, L., Shimkets, R. A., Rossier, B. C., and Lifton, R. P. 1996. Mutations in subunits of the epithelial sodium channel cause salt wasting with hyperkalemic acidosis, pseudohypoaldosteronism type I. Nature Genet. 12:248-253.
Chen, X.-J., Eaton, D. C., and Jain, L. 2002. Beta-adrenergic regulation of amiloride-sensitive lung sodium channels. Am. J. Physiol. (Lung Cell Mol. Physiol.) 282:L609-L620.
Choudhri T F, Hoh B L, Solomon R A, Connolly E S. Jr. and Pinsky D J. 1997 Use of a spectrophotometric hemoglobin assay to objectively quantify intracerebral hemorrhage in mice. Stroke 28: 2296-2302.
Compeau, C. G., Rotstein, O. D., Tohda, H., Marunaka, Y., Rafii, B., Slutsky, A. S., and O'Brodovich, H. 1994. Endotoxin-stimulated alveolar macrophages impair lung epithelial sodium transport by an L-arginine-dependent mechanism. Am. J. Physiol. 266:C1330-C1341.
Delanghe, J R., Duprez, D A., De Buyzere, M L., Bergez, B M., Claeys, L R., Leroux-Roels, G G. and Clement, D L. 1995 Refractory hypertensionis associated with the haptoglobin 2-2 phenotype. J. Cardiovasc Risk, 2:131-6.
Dobryszycka, W. 1997. Biological Function of Haptoglobin-New Pieces to an Old Puzzle. Eur. J. Clin. Chem. Biochem. 35(9):647-654.
Donaldson, S H., Hirsh, A., Li, D C., Holloway, G., Chao, J., Boucher, R C. and Gabriel, S E., Regulation of the epithelial sodium channel by serine proteases in human airways. J Biol. Chem. 2002 Mar. 8:277(10):8338-45.
Garty, H. and Palmer, L. G. 1997. Epithelial sodium channels: Function, structure, and regulation. Physiol. Rev. 77:359-396.
Hardiman, K. M., Lindsey, J. R., and Matalon, S. 2001. Lack of amiloride-sensitive transport across alveolar and respiratory epithelium of iNOS(−/−) mice in vivo. Am. J. Physiol. Lung Cell. Mol. Physiol. 281:L722-L731.
Heinderyckx, M., Jacobs, P., and Bollen, A. 1989 Secretion of glycosylated human recombinant haptoglobin in Baculovirus-infected insect cells. Mol. Biol. Rep. 13:225-232.
Helve, O., Pitkänen, O., A. S., O'Brodovich, H., Kirjavainen, T., and Otulakowski, G. 2004. Low expression of human epithelial Na+ channel (hENaC) in airway epithelium of preterm infants with respiratory distress. Pediatr. 113:1267-1272.
Hummler, E., Barker, P., Gatzy, J., Beermann, F., Verdumo, C., Schmidt, A., Boucher, R., and Rossier, B. C. 1996. Early death due to defective neonatal lung liquid clearance in aENaC-deficient mice. Nature Genet. 12:325-328.
Jain, L., Chen, X. J., Ramosevac, S., Brown, L. A., and Eaton, D. C. 2001. Expression of highly selective sodium channels in alveolar type II cells is determined by culture conditions. Am. J. Physiol Lung Cell Mol. Physiol 280:L646-L658.
Jiang, C., Finkbeiner, W. E., Widdicombe, J. H., McCray, P. B., Jr., and Miller, S. S. 1993. Altered fluid transport across airway epithelium in cystic fibrosis. Science 262:424-427.
Kojima, S., Inenaga, T., Matsuoka, H., Kuramochi, M., Omae, T., Nara, Y. and Yamori, Y. 1994. The association between salt sensitivity of blood pressure and some polymorphic factors. J. Hypertens. 2:797-807.
Knowles, M., Gatzy, J., and Boucher, R. 1981. Increased bioelectric potential difference across respiratory epithelia in cystic fibrosis. N. Engl. J. Med. 305:1489-1495.
Kurosky et al., 1980, Covalent structure of human haptoglobin: a serine protease homolog. Proc. Nat. Acad. Sci. USA, 77, pp. 3388-92.
Langlois M R and Delanghe J R. 1996. Biological and clinical significance of haptoglobin polymorphism in humans. Clin. Chem. 42(10):1589-1600.
Lim, S. K., Kim, H., Lim, S. K., Ali, A. B., Lim, Y. K., Wang, Y., Chong, S. M., Constantini, F., and Baumman, H. 1998. Increased Susceptibility in Hp Knockout Mice During Acute Hemolysis. Blood 92:1870-1877.
Lingueglia, E., Voilley, N., Waldmann, R., Lazdunski, M., and Barbry, P. 1993. Expression cloning of an epithelial amiloride-sensitive Na⁺ channel: A new channel type with homologies to Caenorhabditis elegans degenerins. FEBS Lett. 318:95-99.
Marunaka, Y. and Eaton, D. 1991. Effects of vasopressin and cAMP on single amiloride-blockable Na channels. Am. J. Physiol. Cell Physiol. 260:C1071-C1084.
Matalon, S, and O'Brodovich, H. 1999. Sodium channels in alveolar epithelial cells: Molecular characterization, biophysical properties, and physiological significance. Annu. Rev. Physiol. 61:627-661.
Matthay, M. A., Landolt, C. C., and Staub, N. C. 1982. Differential liquid and protein clearance from the alveoli of anesthetized sheep. J. Appl. Physiol. 53:96-104.
Matsui, H., Grubb, B. R., Tarran, R., Randell, S. H., Gatzy, J. T., Davis, C. W., and Boucher, R. 1998. Evidence for periciliary liquid layer depletion, not abnormal ion composition, in the pathogenesis of cystic fibrosis airways disease. Cell 95:1005-1015.
Matthay, M. A. and Wiener-Kronish, J. P. 1990. Intact epithelial barrier function is critical for the resolution of alveolar edema in man. Am. Rev. Respir. Dis. 142:1250-1257.
Matthay, M. A., Folkesson, H. G., and Clerici, C. 2002. Lung epithelial fluid transport and the resolution of pulmonary edema. Physiol Rev. 82:569-600.
O'Brodovich, H., Canessa, C. M., Ueda, J., Rafii, B., Rossier, B. C., and Edelson, J. 1993. Expression of the epithelial Na+ channel in the developing rat lung. Am. J. Physiol. 265:C491-C496.
O'Brodovich,H.2004. Deficient Na+ channel expression in newborn respiratory distress syndrome. Pediatr. Pulmonol. 26 supplement: 141-142.
Rafii, B., Gillie, D. J., Sulowski, C., Hannam, V., Cheung, T., Otulakowski, G., Barker, P. M., and O'Brodovich, H. 2002. Pulmonary oedema fluid induces non-a-ENaC-dependent Na⁺ transport and fluid absorption in the distal lung. J. Physiol. (Lond.) 544:537-548.
Rossier, B. 2004. The Epithelial Sodium Channel. Proc. Am. Thorac. Soc. 1:4-9.
She, Y., Wang G Q, Loboda, A., Ens W, Standing K G, and Burczynski F J. 2002. Sequencing rat liver cytosolic proteins by MALDI QqTOF mass spectrometry, following electrophoresis separation and extraction. Anal. Biochem. 310:137-147.
Shimkets, R. A., Warnock, D. G., Bositis, C. M., Nelson-Williams, C., Hansson, J. H., Schambelan, M., Gill, J. R. Jr., Ulick, S., Milora, R. V., Findling, J. W. et al. 1994. Liddle's syndrome: Heritable human hypertension caused by mutations in the beta subunit of the epithelial Na channel. Cell 79:404-414.
Tchepichev, S., Ueda, J., Canessa, C. M., Rossier, B. C., and O'Brodovich, H. 1995. Lung epithelial Na channel subunits are differentially regulated during development and by steroids. Am. J. Physiol. 269:C805-C812.
Vallet, V., Chraibi, A., Gaeggeler, H P., Horisberger, J D. and Rossier, B C. 1997. An epithelial serine protease activates the amiloride-sensitive sodium channel. Nature. 389:607-10
Verghese, G. M., Ware, L. B., Matthay, B. A., and Matthay, M. A. 1999. Alveolar epithelial fluid transport and the resolution of clinically severe hydrostatic pulmonary edema. J. Appl. Physiol 87:1301-1312.
Voilley, N., Lingueglia, E., Champigny, G., Mattéi M.-G., Waldmann, R., Lazdunski, M., and Barbry, P. 1994. The lung amiloride-sensitive Na⁺ channel: Biophysical properties, pharmacology, ontogenesis, and molecular cloning. Proc. Natl. Acad. Sci. USA 91:247-251.
Vuagniaux, G., Vallet, V., Jaeger, N F., Pfister, C., Bens, M., Farman, N., Courtois-Coutry, N., Vandewalle, A., Rossier, B C. and Hummler, E. Activation of the amiloride-sensitive epithelial sodium channel by the serine protease mCAP1 expressed in a mouse cortical collecting duct cell line. J. Am. Soc. Nephrol. 2000 May; 11(5):828-34
Ware, L. B. and Matthay, M. A. 2001. Alveolar fluid clearance is impaired in the majority of patients with acute lung injury and the acute respiratory distress syndrome. Am. J. Respir. Crit. Care Med. 163:1383.
Weinberger, M H., Miller, J Z., Fineberg, N S., Luft, F C., Grim, C E. and Christian, J C. 1987. Association of haptoglobin with sodium sensitivity and resistance of blood pressure. Hypertension. 10:443-6
Yue, G. and Matalon, S. 1997. Mechanisms and sequelae of increased alveolar fluid clearance in hyperoxic rats. Am. J. Physiol. 272:L407-L412

1. A method of increasing epithelial Na ion transport and/or epithelial fluid transport in a vertebrate by administering to the vertebrate an effective amount of haptoglobin or an effective amount of haptoglobin and hemoglobin.

2. The method of claim 1 wherein haptoglobin is administered.

3. The method of claim 1 wherein haptoglobin and hemoglobin are administered.

4. The method of claim 3 wherein a haptoglobin/hemoglobin complex is administered.

5. The method of claim 1 wherein the vertebrate is a mammal.

6. The method of claim 5 wherein the mammal is a human.

7. The method of claim 6 wherein the haptoglobin and hemoglobin are human haptoglobin and human hemoglobin.

8. The method of claim 6 wherein the human suffers from a disorder associated with reduced or insufficient Na ion transport or reduced or insufficient Na ion and fluid transport.

9. The method of claim 6 wherein the human suffers from a disorder associated with reduced Na ion transport or excessive fluid accumulation in the lung.

10. The method of claim 8 wherein the human suffers from at least one disorder selected from the group consisting of congestive heart failure-induced pulmonary edema, adult respiratory distress syndrome, neonatal respiratory distress syndrome and transient tachypnea of the newborn.

11. The method of claim 9 wherein the haptoglobin or haptoglobin and hemoglobin is administered by inhalation or instillation into the lung.

12. The method of claim 6 wherein the human suffers from a disorder associated with reduced Na ion transport in the kidney.

13. The method of claim 12 wherein the human suffers from pseudo-hypoaldosteronism or a renal tubular disorder.

14. The method of claim 6 wherein the human suffers from a disorder associated with reduced Na ion transport in the gastro-intestinal tract.

15. The method of claim 14 wherein the human suffers from ulcerative colitis.

16. A pharmaceutical composition comprising haptoglobin or haptoglobin and hemoglobin.

17. The composition of claim 16 wherein the composition comprises a haptoglobin/hemoglobin complex.

18. The composition of claim 16, further comprising a pharmaceutically acceptable carrier.

19. The composition of claim 16, further comprising a pharmaceutically acceptable surfactant.

20. The composition of claim 16, wherein the composition is in the form of an aqueous solution, optionally contained in a device capable of producing an aerosol of the solution.

21. Use of an effective amount of haptoglobin or an effective amount of haptoglobin and hemoglobin in the preparation of a medicament for increasing epithelial Na ion transport in a vertebrate.

22. The use of claim 21 wherein haptoglobin is used.

23. The use of claim 21 wherein haptoglobin and hemoglobin are used.

24. The use of claim 23 wherein a haptoglobin/hemoglobin complex is I used.

25. A method for screening a candidate compound for its ability to modulate haptoglobin or haptoglobin and hemoglobin stimulation of epithelial Na ion transport comprising: providing an in vitro cell system which carries out Na ion transport; contacting the cell system with haptoglobin or haptoglobin and hemoglobin in the presence or absence of the candidate compound; and determining Na ion transport in the cell system; wherein a different level of Na ion transport in the presence of the compound compared with the level in the absence of the compound indicates that the compound modulates the haptoglobin or haptoglobin and hemoglobin stimulation epithelial of Na ion transport.

26. The method of claim 25 wherein the level of Na ion transport in the presence of the candidate compound is lower than the level in the absence of the compound, indicating that the compound inhibits the effect of haptoglobin or haptoglobin and hemoglobin on Na ion transport.
